(19) United States
(12) Patent Application Publication
(10) Pub. No.: US 2019/0183487 A1
(43) Pub. Date: Jun. 20, 2019

(54) DENTAL SUTURING NEEDLE

(71) Applicant: HANMAUM CO.,LTD, Suncheon-si, Jeollanam-do (KR)

(72) Inventor: Nam Gyou KIM, Suncheon-si (KR)

(73) Assignee: HANMAUM CO.,LTD, Suncheon-si, Jeollanam-do (KR)

(21) Appl. No.: 16/306,561

(22) PCT Filed: May 31, 2017
(86) PCT No.: PCT/KR2017/005676

§ 371 Date: Dec. 1, 2018

(30) Foreign Application Priority Data
Jun. 3, 2016 (KR) 10-2016-0069461

(51) Int. Cl. A61B 17/06; A61C 3/00

(52) U.S. Cl. CPC A61B 17/06066; A61B 2017/06028; A61B 2017/06085; A61C 3/00

(57) ABSTRACT

The present invention provides a dental suturing needle comprising a needle part provided with a needle point, and an eye part extending from the needle part and provided with an eye of the needle, wherein between the needle part and eye part is a curved part which is structured so that the needle part slants to the left or right by 20-45° with respect to the eye part. The present invention provides a three-dimensional suturing needle structured so that the needle part curves laterally by a set angle two to three times from the eye part to thus facilitate securing suturing space inside the mouth cavity, remove discomfort to the hand due to easy access to the treatment area, and allow smooth suturing without having to apply excessive pressure to the wrist, and additionally, the present invention is a very useful invention which creates spaces that do not come into contact with tissues to thus allow smooth movement of the suturing needle and minimize damage to the tissues.

### TECHNICAL FIELD

The present invention relates to a dental suturing needle, and more particularly to a three-dimensional suturing needle having a structure wherein the needle part is curved at a predetermined angle in a lateral direction two to three times from the eye part, thereby facilitating securement of a suturing space inside the mouth cavity, allowing easy suturing without stressing the hand and without imposing excessive force on the wrist due to easy access to an area to be treated.

### BACKGROUND ART

As examples of a medical suturing needle used for sealing the living tissue, there are a circular needle with a structure wherein a cross-sectional shape of a part extending from a needle point part to a body part is circular and a cross section of the body part has a circular, triangular, square, or sharp plate shape; and an angular needle with a polygonal shape, such as a triangle, including cutting edges that are formed on a plurality of ridge lines.

An angular needle passes though the living tissue while cutting the living tissue whereas a circular needle, which includes a blunt needle, passes through the living tissue while pushing and opening the living tissue after piercing the living tissue with a needle point thereof.

Accordingly, an optimal suturing needle is selectively used in accordance with the properties of a site to be sutured or other conditions.

When an affected area is sutured, the largest resistance (tingling resistance) is generally generated when a needle point penetrates the living tissue, and thereafter the resistance tends to decrease. Accordingly, research has been conducted to reduce the tingling resistance by sharpening a needle point so as to reduce physical exertion by the doctor and facilitate surgery.

For example, Korean Patent Application Publication No. 10-2010-0051084 (published on May 14, 2010) proposed “medical suturing needle.” This medical suturing needle includes a needle part 2 provided with a needle point 1; and an eye part 3 that is continuously extended, on the same line, from the needle part 2 and is provided with a needle eye 5, and has an arcuate shape overall, as illustrated in FIG. 1.

However, when the conventional medical suturing needle is used inside the mouth cavity or a limited space, it is inconvenient to approach an area to be treated and excessive force is inconveniently applied to the hand or wrist due to difficulties in securing a suturing space because the needle part 2 and the eye part 3 of the medical suturing needle are located on the same line. This may cause tingling resistance increase.

In addition, since the tissue tightly contacts the suturing needle after the needle point 1 passes through the living tissue, it is difficult to smoothly remove the suturing needle from the skin. This may cause tissue damage. Therefore, the present invention is provided to reduce frictional force by providing a non-contact space between a suturing needle and the skin to form an air vent, thereby allowing the suturing needle to easily pass through the tissue.

### DISCLOSURE

### Technical Problem

Therefore, the present invention has been made in view of the above problems, and it is one object of the present invention to provide a three-dimensional suturing needle having a structure wherein the needle part is curved at a predetermined angle in a lateral direction two to three times from the eye part, thereby facilitating securement of a suturing space inside the mouth cavity, allowing easy suturing without stressing the hand and without imposing excessive force on the wrist due to easy access to an area to be treated, and providing a space that is not in contact with the tissue to allow smooth motion of the suturing needle and minimized tissue damage.

### Technical Solution

In accordance with an aspect of the present invention, the above and other objects can be accomplished by the provision of a dental suturing needle including a needle part provided with a needle point; and an eye part continuously extending from the needle part and provided with a needle eye, wherein a curved part is provided between the needle part and the eye part and configured such that the needle part is inclined at an angle of 20 to 45° to the left or right with respect to the eye part.

In addition, the needle part may have a triangular cross section, the eye part may have a semicircular cross section, and a guide groove with a predetermined depth may be respectively formed at the top or bottom of the needle part and the eye part.

### Advantageous Effects

As apparent from the fore-going, the present invention advantageously provides a three-dimensional suturing needle having a structure wherein the needle part is curved at a predetermined angle in a lateral direction two to three times from the eye part, thereby facilitating securement of a suturing space inside the mouth cavity, allowing easy suturing without stressing the hand and without imposing excessive force on the wrist due to easy access to an area to be treated, and providing a space that is not in contact with the tissue to allow smooth motion of the suturing needle and minimized tissue damage.

### DESCRIPTION OF DRAWINGS

FIG. 1 illustrates a front view of a conventional medical suturing needle.

FIG. 2 illustrates a perspective view of a dental suturing needle of the present invention.

FIG. 3 illustrates a side section of a dental suturing needle of the present invention.

FIGS. 4 and 5 illustrate a partial cross-sectional view of the dental suturing needle of FIG. 2.

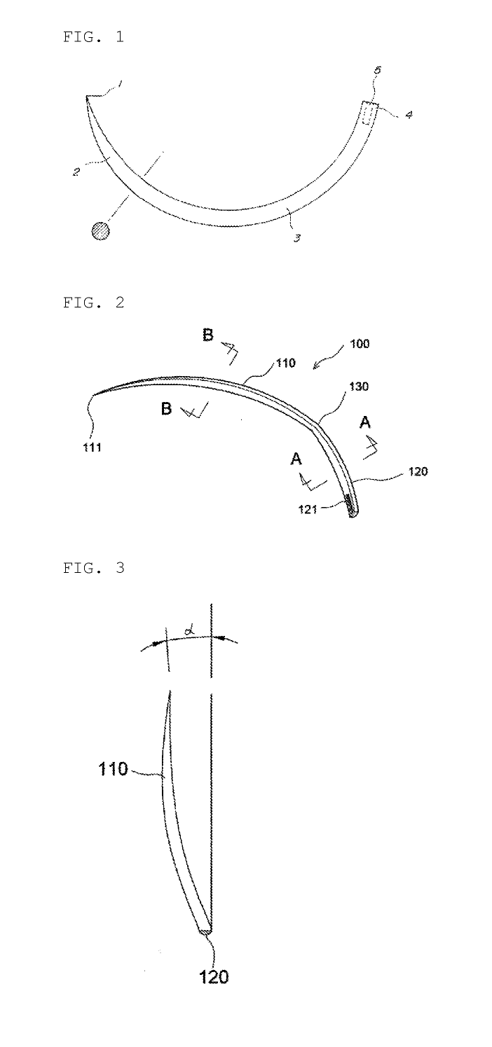

### BEST MODE

Now, preferred embodiment to accomplish the aforementioned purposes and effects of the present invention will be described in more detail.

A dental suturing needle 100 according to the present invention includes a needle part 110 provided with a needle point 111; and an eye part 120 continuously extending from the needle part 110 and provided with a needle eye 121, wherein a curved part 130 is provided between the needle part 110 and the eye part 120 and configured such that the needle part 110 is inclined at an angle of 20 to 45° to the left or right with respect to the eye part 120.

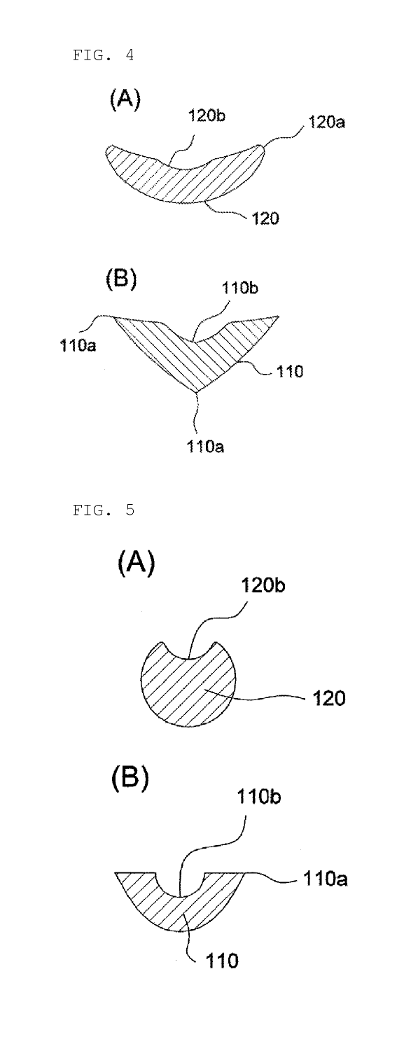

The curved part 130 is formed at a point ⅓ or 3/10 from the needle point 111 or the needle eye 121. The needle point 111 is formed so as to have an inclination (α) of 20 to 45° in a lateral direction (left or right side) with respect to the eye part 120, as illustrated in FIG. 3.

Here, the inclination (α) is preferably 30° to 45°. Since this inclination (α) is an angel providing an approach path at which the hand is most convenient, it is easy to secure a suturing space in the mouth cavity. Accordingly, an affected area can be easily sutured without application of excessive force to the hand or wrist.

Meanwhile, as illustrated in FIG. 4, the needle part 110 has a triangular cross section, the eye part 120 has a semicircular cross section, and guide grooves 110b and 120b having a predetermined depth are respectively formed at the top or bottom of the needle part 110 and the eye part 120.

Here, the needle part 110 has a triangular cross section. This triangular cross section preferably has an end (vertex, 110a) with a sharp shape like a blade so as to facilitate penetration into an area to be treated, e.g., the gum.

In addition, the eye part 120 has a semicircular cross section. The end 120a of the semicircular cross section is preferably curved so as to prevent the living tissue from being torn by the triangular sharp end 110a of the needle part 110 penetrating an area to be treated.

In addition, the guide grooves 110b and 120b with a predetermined depth are respectively formed at the top or bottom of the needle part 110 and the eye part 120, so that the needle part 110 and the eye part 120 can easily pass through the living tissue and enter into the gum through the guide grooves 110b and 120b. Accordingly, a sutured portion can be minimized.

In addition, the needle part 110 may have a semicircular cross section, other than the triangular cross section, as illustrated in FIG. 5. Both ends 110a of the semicircular cross section may have a sharp shape like a blade to facilitate penetration into an area to be treated, i.e., the gum. In addition, the eye part 120 may have a circular cross section.

As described above, since the dental suturing needle 100 of the present invention has the shape wherein the needle part 110 is curved at a predetermined angle in a lateral direction from the eye part 120, it is easy to secure a suturing space inside the mouth cavity and suturing can be easily conducted, without stressing the hand and without imposing excessive force on the wrist, due to easy access to an area to be treated.

*Of course, the dental suturing needle of the present invention is applicable to all medical operations, in which suturing is performed within a limited space, other than the dentistry.

Although the present invention has been described in connection with certain exemplary embodiments, those having ordinary skill in the art to which the present invention pertains will understood that the present invention is not limited to the disclosed exemplary embodiments and various modifications and variations can be made without departing from the scope of the appended claims.

### DESCRIPTION OF SYMBOLS

### *DESCRIPTION OF SYMBOLS CORRESPONDING TO IMPORTANT PARTS OF DRAWING*

100: DENTAL SUTURING NEEDLE OF PRESENT INVENTION

110: NEEDLE PART

110B, 120B: GUIDE GROOVE

120: EYE PART

130: CURVED PART

1. A dental suturing needle, comprising a needle part (110) provided with a needle point (111); and an eye part (120) continuously extending from the needle part (110) and provided with a needle eye (121), wherein a curved part (130) is provided between the needle part (110) and the eye part (120) and configured such that the needle part (110) is inclined at an angle of 20 to 45° to the left or right with respect to the eye part (120).
